(19) United States
(12) Patent Application Publication
(10) Pub. No.: US 2010/0092437 A1
(43) Pub. Date: Apr. 15, 2010

(54) RADIATION INDUCIBLE PROMOTER

(75) Inventors: SANGYONG LIM, JEONGEUP-SI (KR); JINWOO JUNG, DAEJEON (KR); JUNG-MU HUR, JEONGEUP-SI (KR); DONGHO KIM, JEONGEUP-SI (KR); MINHO JOE, JEONGEUP-SI (KR)

Correspondence Address: GREENLEE WINNER AND SULLIVAN P C, 4875 PEARL EAST CIRCLE, SUITE 200, BOULDER, CO, 80301, US

(73) Assignee: KOREA ATOMIC ENERGY RESEARCH INSTITUTE, DAEJEON (KR)

(21) Appl. No.: 12/361,142

(22) Filed: Jan. 28, 2009

(30) Foreign Application Priority Data
Oct. 9, 2008 (KR) 1020080099124

(51) Int. Cl. A61K 35/12; C12N 15/11; C12N 15/00; C12N 1/21; C12P 21/00; A61K 35/00

(52) U.S. Cl. 424/93.21; 536/24.1; 435/320.1; 435/252.33; 435/69.1; 424/93.2

(57) ABSTRACT

The present invention relates to a radiation inducible promoter, more particularly to a radiation inducible promoter including a base sequence represented by SEQ ID NO: 3 derived from Salmonella typhimurium, an expression vector including the promoter, a transformant including the expression vector, and a method for inhibiting and treating cancer using the transformant. Because the radiation inducible promoter of the present invention may control the amount of a target protein produced by radiation, problems such as cytotoxicity caused by overproduction of an anticancer agent in the cancer cell may be solved and the anticancer agent may be used as an effective treatment simultaneously with a radiation therapy due to production of the agent in a cancer cell.

### BACKGROUND OF THE INVENTION

1. Field of the Invention

The present invention relates to a radiation inducible promoter, more particularly to a radiation inducible promoter including a base sequence represented by SEQ ID NO: 3 derived from Salmonella typhimurium, an expression vector including the promoter, a transformant including the expression vector, and a method for inhibiting and treating cancer using the transformant.

2. Description of the Related Art

A radiation anticancer therapy is combined with drug treatment or used to prevent metastasis of cancer after operation. Recently, various methods for treating cancer have been developed to minimize side effects of radiation and increase treatment efficiency.

Cytokines such as tumor necrosis factor (TNF), interleukins (ILs), and interferons (IFs) have been used as anticancer agents, but they have problems that they show cytotoxicity when treated in excess and are diluted in blood when administered in small amounts. In order to overcome these problems, a gene therapy whereby genes for producing the anticancer agents are delivered into cancer cells using a gene transfer vector or a gene delivery system, and then cytokines are produced in the cancer cells to treat the cancer has been developed. However, when the anticancer agents are produced by promoters such as constitutive promoters which may not control the expression, the anticancer agents may have problems caused by overproduction in the cancer cell, and may cause severe side effects when delivered into non-cancerous tissues. Thus, it is more efficient to produce anticancer agents using inducible promoters which may regulate the production of anticancer agents temporally and spatially.

A protein expression system utilizing a radiation inducible promoter has an advantage that anticancer agents may be produced in the cancer cell simultaneously with a radiation therapy. Various methods for treating cancer, using gene transfer vectors including radiation responsive promoters have been developed. Among them, the most widely known method is the one to kill cancer cells by delivering a radiation inducible promoter known as an EGR1 and a TNF-αDNA complex into the cancer cell, using an adenovirus, a kind of gene transfer vector, and then increasing the production of TNF-α by radiation (See U.S. Pat. No. 6,156,736).

Gene carriers developed so far are the following: 1) Viral vectors using retroviruses and adenoviruses, 2) Non-viral vectors called nano-particles, and 3) Bacterial vectors using anaerobic microorganisms such as Clostridium, Bifidobacterium, and Salmonella (See Table 1).

TABLE 1

| | | Non-viral | Bacterial |
|---|---|---|---|
| | Viral vectors | vectors | vectors |
| Stability | + | +++ | + |
| Efficiency | +++ | + | + |
| Low production | + | ++ | +++ |
| cost | | | |
| Simple | + | ++ | +++ |
| production | | | |
| Simple | ++ | + | +++ |
| transportation | | | |
| Amount of DNA | ++ | + | +++ |
| transferred | | | |

Until now, radiation inducible promoters used in anticancer treatments have been used inherently as gene transfer vectors because most of the promoters are the promoters of animal cell genes. As known in Table 1, bacterial vectors have more advantages compared to viral vectors, but they may not be used in the anticancer gene therapy by radiation because good radiation inducible promoters have not been discovered. In the past, researches on construction of Clostridium-based gene therapy system by increasing the production of TNF-α, using radiation inducible promoters of Clostridium were conducted (Nuyts S, et al., Anti-Cancer Drugs. 13(2):115-125, February 2002), but there is a need for development of more various and effective radiation inducible promoters.

Thus, the present inventors developed an expression regulatory system using a radiation-dependent promoter through a radiation inducible promoter derived from Salmonella which is not an animal cell, but a bacteria cell, confirmed that an exogenous protein expression might be regulated by irradiation using the expression regulatory system, and discovered that ultimately anticancer agents in cancer cells might be produced at the same time with a radiation therapy, leading to the completion of the present invention.

### SUMMARY OF THE INVENTION

### Technical Problem

An object of the present invention is to provide a new radiation inducible promoter, a gene construct in which an exogenous protein is operably linked to the promoter, an expression vector including the gene construct, a transformant transformed with the expression vector, and a method for inhibiting and treating cancer including irradiating the transformant.

### Technical Solution

In order to accomplish the above object, the present invention provides a radiation inducible promoter including a base sequence represented by SEQ ID NO: 3.

The present invention also provides an expression vector including the promoter and a transformant transformed with the expression vector.

The present invention also provides a gene construct in which a gene encoding an exogenous protein is operably linked to the promoter, an expression vector including the gene construct, and a transformant transformed with the expression vector.

The present invention also provides a method for producing an exogenous protein, including irradiating the transformant after being cultured.

The present invention also provides a composition for inhibiting and treating cancer, including a recombinant vector in which a gene encoding an anticancer protein is operably inserted into an expression vector including the gene construct, and a transformed cell with the recombinant vector.

The present invention also provides a method for inhibiting and treating cancer, including administering the recombinant vector or a transformed cell with the recombinant vector to an individual and irradiating the individual.

Furthermore, the present invention provides a use of a recombinant vector in which a gene encoding an anticancer protein is operably inserted into the expression vector including the gene construct, or a transformed cell with the recombinant vector in manufacture of a composition for inhibiting and treating cancer.

Hereinafter, the present invention will be described in detail.

Unless otherwise specifically indicated, the terms and technologies as described herein will be interpreted to be those used in the art to which the present invention pertains.

The present invention provides a radiation inducible promoter including a base sequence represented by SEQ ID NO: 3.

The radiation inducible promoter is preferably derived from, but not limited to, Salmonella typhimurium, either naturally-derived or artificially synthesized.

The radiation inducible promoter is positioned between the cyoA gene and the ampG gene, and preferably transcribed in the opposite direction (3′ to 5′) to the direction (5′ to 3′) of transcription of the cyoA gene and the ampG gene but not limited to this.

The radiation inducible promoter has preferably a base sequence represented by SEQ ID NO: 3, but any base sequence which has at least 70% or higher homology to the base sequence and the same function will be included in the present invention.

Because the radiation inducible promoter does not form an open reading frame (ORF) in which a start codon and a stop codon exist, and does not synthesize a protein, it may preferably produce only a mRNA, but is not limited to this.

In the radiation inducible promoter, it is preferable that an RNA polymerase binding site exists between 203rd and 318th nucleotide in a base sequence represented by SEQ ID NO: 2 and a radiation response element exists between 318th and 461st nucleotide, but is not limited to this.

After analyzing the base sequence of a Salmonella typhimurium gene, the present inventors discovered a genetic site which was positioned between the cyoA gene and the ampG gene, transcribed in the opposite direction (3′ to 5′) to the direction of transcription of the cyoA gene and the ampG gene, and did not form an open reading frame (See FIG. 1). The sequence of the DNA fragment was described as SEQ ID NO: 1 and the DNA fragment was referred as “uscA (upstream of cyoA)” (See FIG. 2).

The present inventors amplified the DNA fragment represented by SEQ ID NO: 2 with a primer, and then cloned it into a reporter vector having β-galactosidase producing gene. Specifically, a pUscA-1 vector is prepared to include a DNA fragment amplified with uscA5 and uscA6 primers, a pUscA-2 vector to include a DNA fragment amplified with uscA7 and uscA6 primers, a pUscA-3 vector to include a DNA fragment amplified with uscA5 and uscA8 primers, a pUscA-4 vector to include a DNA fragment amplified with uscA7 and uscA8 primers, a pUscA-5 vector to include a DNA fragment amplified with uscA7 and uscA9 primers, a pUscA-6 vector to include a DNA amplified with uscA10 and uscA8 primers and a pUscA-7 vector to include a DNA amplified with uscA10 and uscA6 primers, respectively. After each of the seven transformants prepared above was cultured respectively, the amounts of β-galactosidase produced before and after radiation were measured according to the Miller method (Experiments in molecular genetics, Cold Spring Harbor Lab., 1972). The results showed that the pUscA-5 vector didn't produce β-galactosidase regardless of radiation at all but pUscA-3, pUscA-4, and pUscA-6 vectors produced β-galactosidase even in the absence of radiation, and the productions were not increased even by radiation. However, pUscA-1, pUscA-2, and pUscA-7 vectors produced low levels of β-galactosidase before radiation and produced large amounts after radiation. Thus, it was realized that promoter-factors such as −35 and −10 regions of the uscA promoters (RNA polymerase binding sites) were between 203rd and 318th nucleotide in the DNA fragment represented by SEQ ID NO: 2 and the radiation response elements of the uscA promoter were between 318th and 461st nucleotide (See FIG. 5 and FIG. 6). It is to be understood that 203rd and 461st nucleotides represented by SEQ ID NO: 3 in the DNA fragment represented by SEQ ID NO: 2 are the core sites.

When the transcription start site was explored using a primer extension method, it was realized that the transcription of the uscA started in the 282nd nucleotide represented by SEQ ID NO: 2. To sum up, it is to be understood that a repressor which inhibits the expression between 318th and 461st nucleotide of the uscA promoter is bound but when irradiated the repressor drops from the promoter to induce the expression of the uscA promoter.

The present invention also provides an expression vector including a radiation inducible promoter including a base sequence represented by SEQ ID NO: 3 and a transformant transformed with the expression vector.

The present invention also provides a gene construct in which an exogenous protein is operably linked to the radiation inducible promoter including a base sequence represented by SEQ ID NO: 3, an expression vector including the gene construct, and a transformant transformed with the expression vector.

The gene construct may be used in preparation of a recombinant vector with a structure consisting of a structural gene encoding an exogenous protein operably linked to the promoter and a transcription terminator. It is also possible to insert the construct into a commercially available vector according to genetic engineering techniques and use it.

It is convenient for those skilled in the art to insert a gene encoding an exogenous protein which they seek to express into the expression vector using multiple-cloning sites. It is also easy for them to screen the gene using antibiotic resistance genes. The multiple-cloning sites are preferably selected by using base sequences known to those skilled in the art and the antibiotic resistance genes also known to those skilled in the art are preferably used.

The exogenous protein is preferably selected from the group consisting of, but not limited to, hormones, hormone analogues, enzymes, enzyme inhibitors, receptors, portions thereof, antibodies and portions thereof, monoclonal antibodies, structural proteins, toxic proteins, and plant defense activators.

A basis vector for preparation of the expression vector is preferably, but not limited to, a pGL3 vector, and any vector may be used if it stably exists in bacteria and is high in copy number.

A host strain for transforming the expression vector is preferably, but not limited to, E. coli, and any bacteria is possible if it has an RNA polymerase which recognizes the promoter of the present invention.

The present inventors amplified an about 500 bp DNA fragment (SEQ ID NO: 2)—between XhoI and HindIII sites—in a base sequence represented by SEQ ID: 1 with PCR, and then prepared a recombinant expression vector (referred to as “pGLR”) by cloning the fragment in the pGL3 vector which has a luciferase production gene commonly used as a reporter protein. A transformant including a radiation inducible promoter was prepared by transforming the recombinant expression vector in E. coli (See FIG. 3).

In addition, the present invention provides a method for producing an exogenous protein, including:

1) culturing a transformant transformed with an expression vector including a gene construct in which a gene encoding the exogenous protein is operably linked to a radiation inducible promoter including a base sequence represented by SEQ ID NO: 3; and

2) irradiating the cultured transformant in step 1).

In the method, a radiation source in the radiation in step 2) is selected preferably from the group consisting of gamma-ray, electron beam, and X-ray, and more preferably gamma-ray, but not limited to this (Dauphin J F et al., Elsevier Scientific, pp. 131-220). The gamma-ray is the one preferably emitted from a radioactive isotope such as Co-60, Kr-85, Sr-90, or Cs-137, and more preferably from Co-60, but not limited to this. An absorbed dose of the radiation is preferably 1 to 5 Gy, and more preferably 2 to 4 Gy, but not limited to this.

The present invention also provides a composition for inhibiting and treating cancer, including a recombinant vector in which a gene encoding an anticancer protein is operably inserted into an expression vector including a gene construct in which a gene encoding an exogenous protein is operably linked to a radiation inducible promoter including a base sequence represented by SEQ ID NO: 3, or a cell transformed with the recombinant vector.

The anticancer protein is preferably a cytokine, and the cytokine is preferably, but not limited to, a tumor necrosis factor (TNF), interleukins (ILs), or interferons (IFs).

The cell is preferably, but not limited to, an allogenic cell or an autologous cell.

The present invention also provides a method for inhibiting and treating cancer, including:

1) preparing a recombinant vector by operably inserting a gene encoding an anticancer protein into an expression vector including a gene construct in which a gene encoding an exogenous protein is operably linked to a radiation inducible promoter including a base sequence represented by SEQ ID NO: 3;

2) administering the recombinant vector in step 1) or a cell transformed with the recombinant vector into an individual; and

3) irradiating the individual in step 2).

In the method, the expression vector in step 1) is preferably, but not limited to, a pGL3 vector, and any vector may be used if it stably exists in bacteria and high in copy number.

In the method, the anticancer protein in step 1) is preferably a cytokine, and more preferably, but not limited to, a tumor necrosis factor (TNF), interleukins (ILs), or interferons (IFs), and any protein is possible if it has anticancer effects.

In the method, the cell in step 2) is preferably, but not limited to, an allogenic cell, an autologous cell, or a cancer cell.

In the method, a radiation source in the radiation in step 3) is selected preferably from the group consisting of gamma-ray, electron beam, or X-ray, and more preferably gamma-ray, but not limited to this (Dauphin J F et al., Elsevier Scientific, pp. 131-220). The gamma-ray is one preferably emitted from a radioactive isotope such as Co-60, Kr-85, Sr-90, or Cs-137, and more preferably from Co-60, but not limited to this. An absorbed dose of the radiation is preferably 1 to 5 Gy, and more preferably 2 to 4 Gy, but not limited to this.

In order to observe whether the amount of an exogenous protein produced by using a radiation inducible promoter whose expression was induced by radiation might be controlled, the present inventors inoculated the transformant prepared above into a medium, irradiated and cultured it at various absorbed doses, and measured an expression level of luciferase, a target protein, by using a luminometer. As a result, in case of an irradiation at 2 to 4 Gy on the transformant of the present invention, the amount of luciferase produced was increased by about 5 to 100 times compared to a non-irradiated control group. In particular, in case of an irradiation at 2 Gy, the amount was increased by about 100 times (See FIG. 4).

Thus, a method for inhibiting and treating cancer by using a radiation inducible promoter of the present invention allows a radiation therapy to be performed simultaneously with an anticancer therapy by producing an anticancer protein in the cancer cell through radiation.

Furthermore, the present invention provides a use of a recombinant vector in which a gene encoding an anticancer protein is operably inserted into an expression vector including the gene construct or a cell transformed with the recombinant vector in manufacture of a composition for inhibiting and treating cancer.

The anticancer protein is preferably a cytokine, and the cytokine is preferably, but not limited to, a tumor necrosis factor (TNF), interleukins (ILs), or interferons (IFs).

The cell is preferably, but not limited to, an allogenic cell or an autologous cell.

Because a radiation inducible promoter of the present invention may control the amount of a target protein produced by radiation, a problem caused by the overproduction of a target protein may be solved, the production of a protein showing cytotoxicity may be controlled, and a target protein, when used with an anticancer substance as a therapeutic agent, may be used as an effective treatment method simultaneously with a radiation therapy.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a diagram showing the position and transcription direction of a radiation inducible genome (uscA) in a Salmonella typhimurium gene;

FIG. 2 is a diagram showing base sequences positioned between the cyoA gene including a radiation inducible uscA promoter in a Salmonella typhimurium gene and the ampG gene;

FIG. 3 is a diagram showing the procedure in which a radiation inducible uscA promoter prepares a cloned vector (pGLR);

FIG. 4 is a graph showing the change of the amount of luciferase produced after a transformant introduced by a vector (pGLR) in which a radiation inducible promoter was cloned was irradiated at various absorbed doses;

FIG. 5 is a diagram showing a DNA fragment of a radiation inducible uscA promoter cloned in the pRS415 vector, a reporter vector, and the activity of β-galactosidase produced from each vector after an irradiation at 2 Gy; and

FIG. 6 is a diagram showing a transcription start site of a uscA promoter, and −35 and −10 promoter factors bound with an RNA polymerase.

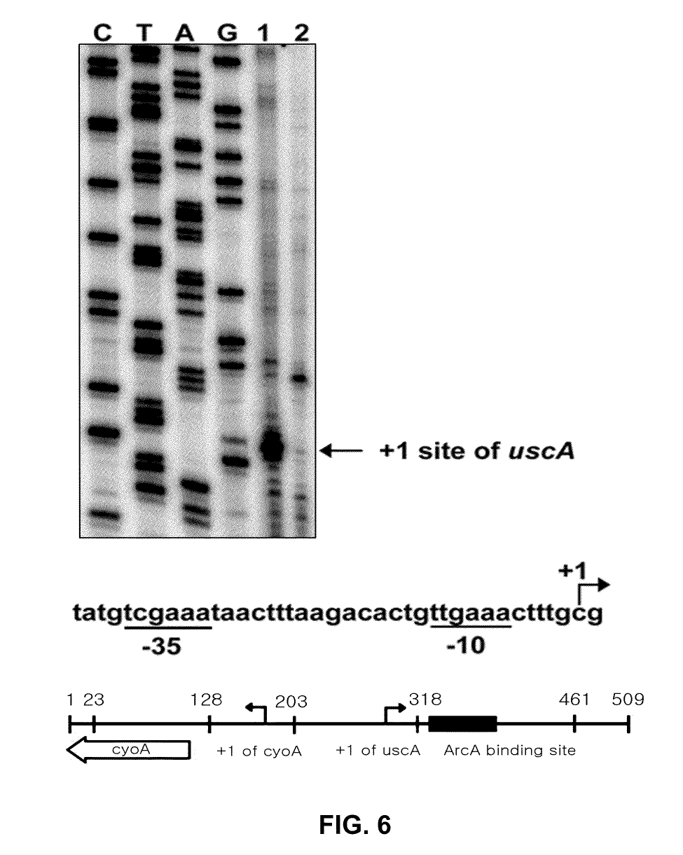

### DESCRIPTION OF THE PREFERRED EMBODIMENTS

Hereinafter, the present invention will be described in detail with reference to embodiments.

However, the following embodiments are provided only to illustrate the present invention, and the present invention is not limited to them.

### Embodiment 1

### Analysis of a Radiation Inducible Promoter

The present inventors inoculated Salmonella strains (Salmonella enterica serovar Typhimurium SL1344) into an LB medium (distilled water, 1 L; tryptone, 10 g; yeast extract, 5 g; NaCl, 10 g were mixed, dissolved, and autoclaved at 121° C. for 20 min) and shaking-cultured the mixture at 37° C. Genomic DNAs were recovered from the SL1344 strains under the conditions using a DNeasy Tissue kit (Qiagen, Hilden, Germany), and then a target DNA fragment was amplified using a Primus 96 thermal cycler (MWG Biotech Inc., High Point, N.C., USA). PCR conditions were as the followings. \textcircled{1} PCR reaction solution: genomic DNA, 100 ng; each 50 pmole of uscA1 and uscA2 primers (Table 1); AccuPower HF PCR premix (Bioneer Co. Korea) \textcircled{2} PCR reaction conditions: repeating the steps of denaturation (94□, 30 sec), annealing (50□, 30 sec), and extension (72□, 30 sec) 40 times. The amplified DNA fragments were purified using a PCR DNA purification kit (GeneAll Biotech., Korea), recovered, and sent to Xenotech (Daejeon, Korea) for analysis of base sequences. The analysis of base sequences was performed using an ABI3730xl sequencer (Applied Biosystems Inc., CA, USA). In addition, the transcription direction of a target gene was identified by confirming that the expression was induced by radiation when a DNA fragment with a cyoA promoter site was cloned into a pGL3 vector in the opposite direction to the transcription direction of the cyoA.

TABLE 1

| | |
|---|---|
| Primer | Base sequence (5′ → 3′) |

| | |
|---|---|
| uscA1 | atc aac atc agg cca aat gc |
| | (SEQ ID NO: 4) |
| uscA2 | taa taa cga ccc gga att at |
| | (SEQ ID NO: 5) |
| uscA3 | tag ccc ggg ctc gag¹ tcc aat ctg |
| | (SEQ ID NO: 6) |
| uscA4 | acg gct aaa taa gct t²ca att gat |
| | (SEQ ID NO: 7) |
| uscA5 | aat ctg tcc ttt gga att c³ag |
| | (SEQ ID NO: 8) |
| uscA6 | gca aca gaa gga tcc⁴ aaa tta |
| | (SEQ ID NO: 9) |
| uscA7 | taa cga cct gaa ttc³ cac ggg acc |
| | (SEQ ID NO: 10) |
| uscA8 | ata cca agg atc c⁴ct caa cat |
| | (SEQ ID NO: 11) |
| uscA9 | tgg taa cac gga tcc⁴ aat cat gtt |
| | (SEQ ID NO: 12) |
| uscA10 | aac atg att gaa ttc³ gtg tta cca |
| | (SEQ ID NO: 13) |

¹XhoI site;
²HindIII site;
³EcoRI site;
⁴BamHI site

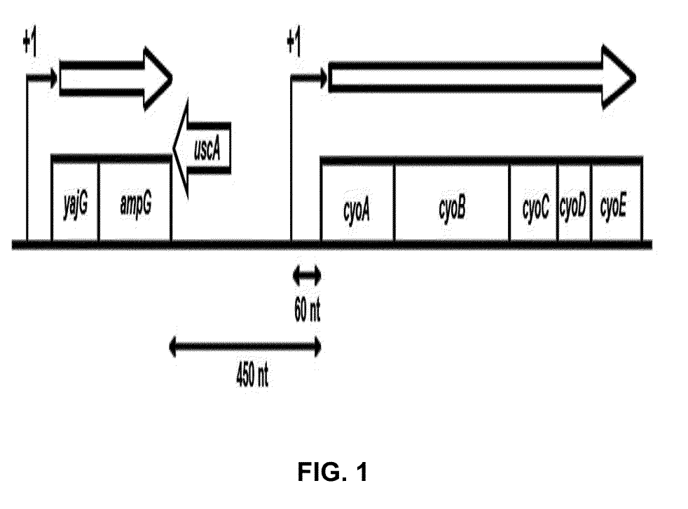

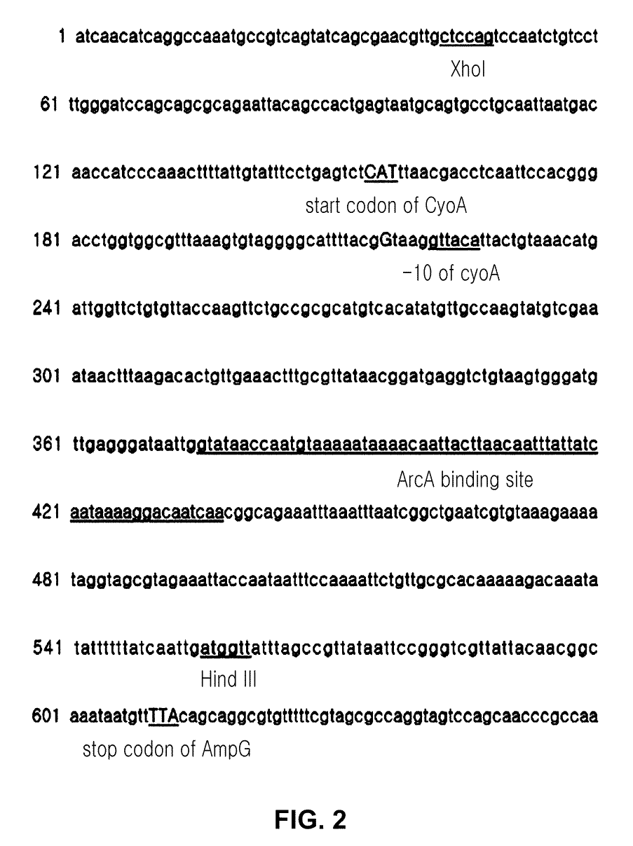

The analysis results showed that as illustrated in FIG. 1, the target gene was positioned between the cyoA gene and the ampG gene, and a gene site which was transcribed in the opposite direction (3′ to 5′) to the transcription directions of the cyoA gene and the ampG gene (5′ to 3′) was found (See FIG. 1). Because the gene site failed to form an open reading frame (ORF) in which a start codon where the translation starts and a stop codon where the translation terminates exist, it was a DNA fragment which could not synthesize a protein and might produce only an mRNA. The DNA fragment is described as SEQ ID NO: 1 (FIG. 2). The present inventors referred to the DNA fragment as “uscA (upstream of cyoA).

### Embodiment 2

### Preparation of an Expression Vector and a Transformant

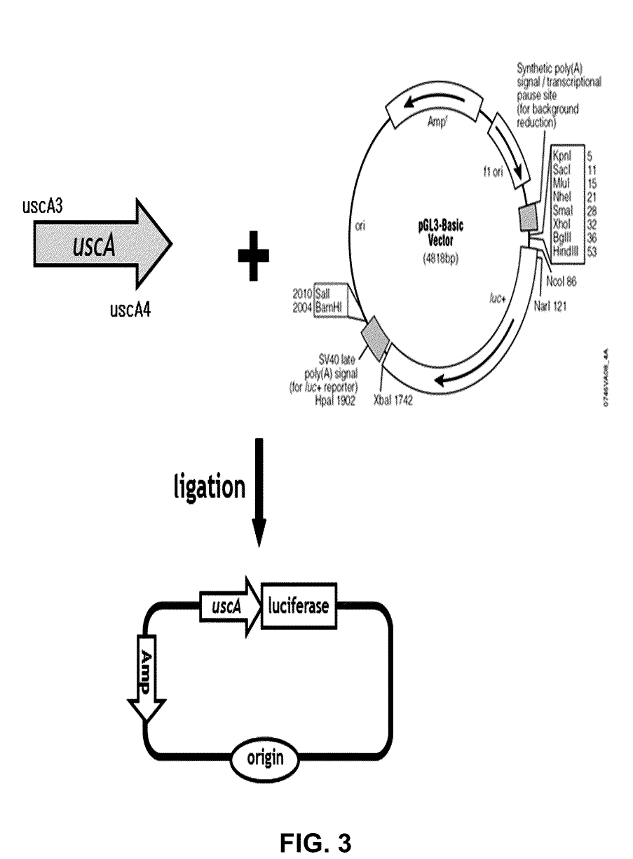

The present inventors amplified the DNA fragment represented by SEQ ID NO: 1 with a primer having XhoI and HindIII restriction enzyme recognition sites (uscA3 and uscA4: Table 1). PCR was performed in the same way as in <Embodiment 1>. By treating both the ends of the amplified DNA fragment with XhoI and HindIII restriction enzymes, the DNA fragment represented by SEQ ID NO: 2 (between XhoI and HindIII sites) was cloned into a purchased pGL3 vector (promega, USA). The pGL3 vector has a luciferase production gene used as a reporter protein. A transformant was prepared by transforming the cloned vector (hereinafter, referred to as “pGLR”) in E. coli JM109 (See FIG. 3). In the transformation, the preparation and transformation of competent cells of E. coli JM 109 was performed using the method described in Goodman et al., Methods Enzymol., 68:75-90, 1979. In order to identify the transformation, the transformant was cultured in an LB medium supplemented with ampicilin (100 μg/M [symbol] ), and then the pGLR was recovered and the base sequence was analyzed (Xenotech; Daejeon, Korea) using a plasmid prep kit (GeneAll Biotech., Korea).

### Embodiment 3

### Identification of the Amount of a Target Protein Produced by Radiation

The present inventors had shaking-cultured the transformant prepared in <Embodiment 2> in the LB medium at 37□ for 12 hours. Then gamma-ray was irradiated on the medium using a Cobalt 60-gamma irradiator (point source; IR-79, AECL, Ottawa, ON, Canada) in Advanced Radiation Technology Institute, Korea Atomic Energy Research Institute, Jeongeup. The dose rate was 1 Gy/min and irradiation doses at 0, 2, 4, 6, 8, and 10 Gy were irradiated. After the irradiation, the medium was cultured at 37□ in an incubator for 1 hour. The medium was centrifuged (10 min, 10000 r.p.m., 4□), the supernatant was removed, and the expression of luferase for the precipitate was measured using a Luciferase assay system (Promega, USA) and luminometer (Berthold Tech., UK).

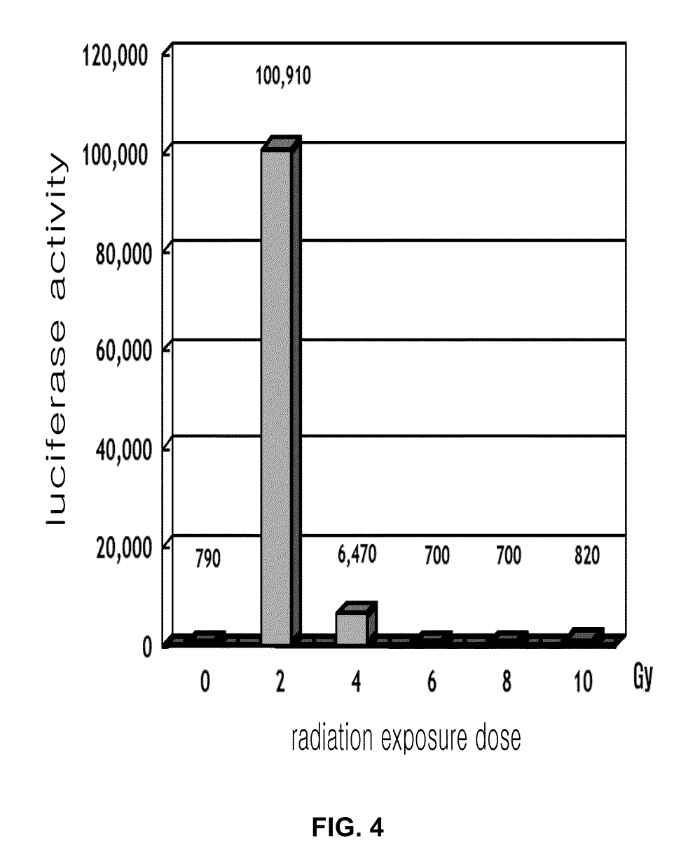

As a result, it was realized that when radiation at 2 to 4 Gy was irradiated on the transformant of the present invention, the amount of luciferase produced was increased by about 5 to 130 times compared to a non-irradiated control group, and that especially in case of irradiation at 2 Gy, the amount of luciferase produced was increased by 100 times compared to a non-irradiated control group (See FIG. 4).

### Embodiment 4

### Discovery of Radiation Response Elements of a uscA Promoter

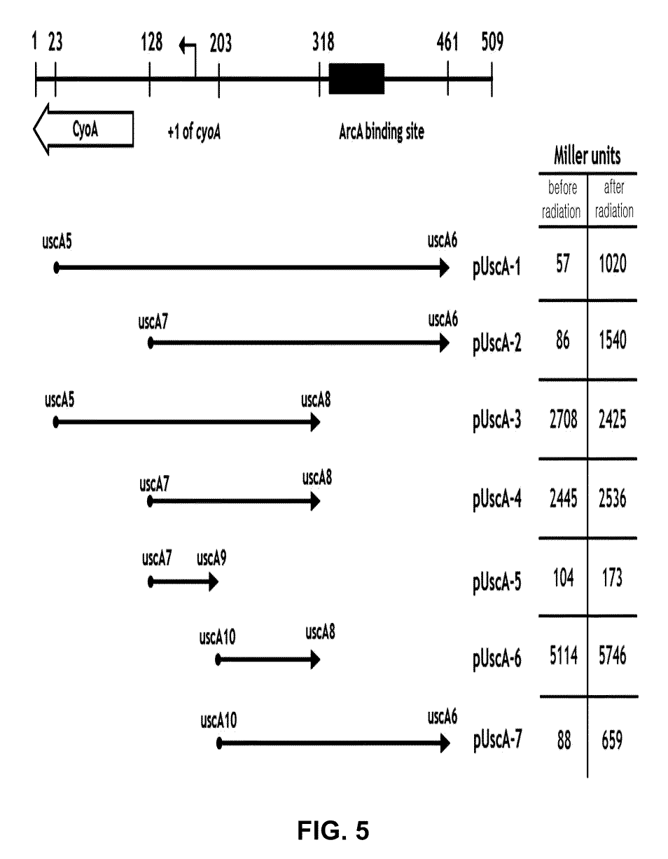

The present inventors amplified the DNA fragment represented by SEQ ID NO: 2 with primers having EcoRI and BamHI restriction enzyme recognition sites (uscA5 to uscA10; Table 1). PCR was performed in the same way as in <Embodiment 1>. Both the ends of the amplified DNA fragments with various lengths was treated with EcoRI and BamHI restriction enzymes and cloned into a pRS415 vector (NCBI accession No. U03449). The pRS415 vector has a β-galactosidase production gene as a lacZ reporter vector. The pUscA-1 vector was treated to include a DNA fragment amplified with uscA5 and uscA6 primers, the pUscA-2 vector with uscA7 and uscA6 primers, the pUscA-3 vector with uscA5 and uscA8 primers, the pUscA-4 vector with uscA7 and uscA8 primers, the pUscA-5 with uscA7 and uscA9 primers, the pUscA-6 vector with uscA10 and uscA8 primers, and the pUscA-7 vector with uscA10 and uscA6 primers, respectively (See FIG. 5). The preparation and identification of the transformant of the cloned vector was performed in the same way as in <Embodiment 2>. Each of the four kinds of the transformants was cultured in the LB medium at 37□ for 3 hours, was irradiated at 2 Gy as in <Embodiment 3>, and then the amounts of β-galactosidase produced before and after radiation was measured using the Miller method (Experiments in molecular genetics, Cold Spring Harbor Lab., 1972).

As a result, the pUscA-5 didn't produce β-galactosidase regardless of irradiation at all but pUscA-3, pUscA-4, and pUscA-6 vectors produced β-galactosidase even in the absence of irradiation, and the productions were not increased even by radiation. However, pUscA-1, pUscA-2, and pUscA-7 vectors produced low levels of β-galactosidase before radiation and produced large amounts after radiation (about 10 to 20 times increase compared to before radiation).

Based on the results, it was realized that promoter-factors such as −35 and −10 regions of the uscA promoter (RNA polymerase binding sites) were between 203rd and 318th nucleotide in the DNA fragment represented by SEQ ID NO: 2 and the radiation response elements of the uscA promoter were between 318th and 461st nucleotide. In conclusion, it is to be understood that a repressor which inhibits the expression between 318th and 461st nucleotide of the uscA promoter is bound but when irradiated the repressor drops from the promoter to induce the expression of the uscA promoter.

### Embodiment 5

### Discovery of Transcription Start Site of the uscA Promoter

The present inventors explored transcription start site using a transformant of the pUscA-3 vector used in <Embodiment 4>. The pUscA-3 transformant was shaking-cultured in 50 M [symbol] LB medium for 3 hours (lane 1) or standing-cultured (lane 2), and then a total RNA was recovered with a TRIZOL reagent (GIBCO BRL). A primer having a complementary sequence (5′-att aac tgc gcg tcg ccg ctt tca tcg gtt-3′) (SEQ ID NO: 14) to a base sequence in the BamHI downstream of the pRS415 vector was marked as [γ-³²P]-ATP using a polynucleotide kinase (PNK) (Invitrogen) and then mixed with 30 μg of the recovered total RNA. And then a cDNA was synthesized from the total RNA using a reverse transcriptase (Superscript II; Invitrogen) and a transcript was identified from the electrophoresis of the synthesized cDNA in 6% acrylamide gel. A DNA base sequence ladder for identification of the transcription start site was produced using a primer marked with the isotope and a pUscA-4 vector as a PCR primer and a substrate, respectively through a SequiTherm EXCEL II DNA Sequencing Kit (EPICENTRE Biotech.), and then was electrophoresized with the cDNA.

As a result, it was realized that the transcription of the uscA was started in the 282nd nucleotide of the fragment represented by SEQ ID NO: 2, and −35 and −10 regions of the uscA promoter (RNA polymerase binding sites) were presumed.

### ADVANTAGEOUS EFFECTS

The radiation inducible promoter of the present invention may be used in a method for overproducing an anticancer agent in cancer cells and used for inhibiting and treating cancer, and be useful as an effective method for treating cancer due to production of an anticancer agent in cancer cells, simultaneously with a radiation therapy.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

We claim:

1. A radiation inducible promoter comprising a base sequence represented by SEQ ID NO: 3.

2. The radiation inducible promoter as set forth in claim 1, wherein the promoter is derived from Salmonella typhimurium.

3. The radiation inducible promoter as set forth in claim 1, wherein the promoter is positioned between the cyoA gene and the ampG gene.

4. The radiation inducible promoter as set forth in claim 1, wherein the promoter is transcribed from right to left.

5. The radiation inducible promoter as set forth in claim 1, wherein an RNA polymerase binding site in the promoter exists between 203rd and 318th nucleotide in the base sequence represented by SEQ ID NO: 3.

6. The radiation inducible promoter as set forth in claim 1, wherein a radiation response element in the promoter exists between 318th and 461st nucleotide in the base sequence represented by SEQ ID NO: 3.

7. An expression vector comprising a radiation inducible promoter having a base sequence represented by SEQ ID NO: 3.

8. A transformant transformed with the expression vector of claim 7.

9. A gene construct in which a gene encoding an exogenous protein is operably linked to a radiation inducible promoter having a base sequence represented by SEQ ID NO:3.

10. An expression vector comprising the gene construct of claim 9.

11. A transformant transformed with the expression vector of claim 10.

12. A method for producing an exogenous protein, comprising:
1) culturing the transformant of claim 8 or claim 11; and
2) irradiating the transformant cultured in step 1.

13. A composition for inhibiting and treating cancer, comprising a recombinant vector in which a gene encoding an anticancer protein is operably inserted into the expression vector of claim 7 or claim 10, or a cell transformed with the recombinant vector.

14. The composition as set forth in claim 13, wherein the anticancer protein is a cytokine.

15. The composition as set forth in claim 14, wherein the cytokine is a tumor necrosis factor (TNF), interleukins (ILs), or interferons (IFs).

16. The composition as set forth in claim 13, wherein the cell is an allogenic cell or an autologous cell.

17. A method for inhibiting and treating cancer, comprising:
1) preparing a recombinant vector by operably inserting a gene encoding an anticancer protein into the expression vector of claim 7 or claim 10;
2) administering a cell transformed with the recombinant vector in step 1 or the recombinant vector to an individual; and
3) irradiating the individual in step 2.

18. The method as set forth in claim 17, wherein the cell in step 2 is an allogenic cell, an autologous cell, or a cancer cell.

19. The method as set forth in claim 17, wherein the radiation in step 3 is one selected from the group consisting of gamma-ray, electron beam, and X-ray.

20. The method as set forth in claim 19, wherein the radiation is gamma-ray.

21. The method as set forth in claim 20, wherein the gamma-ray is emitted from one radioactive isotope selected from the group consisting of Co-60, Kr-85, Sr-90, and Cs-137.

22. The method as set forth in claim 17, wherein an absorbed dose of the radiation in step 3) is 1 to 5 Gy.

23. A use of a cell transformed with a recombinant vector in which a gene encoding an anticancer protein is operably inserted into the expression vector of claim 7 or claim 10, or the recombinant vector, in manufacture of a composition for inhibiting and treating cancer.

24. The use as set forth in claim 23, wherein the anticancer protein is a cytokine.

25. The use as set forth in claim 24, wherein the cytokine is a tumor necrosis factor (TNF), interleukins (ILs), and interferons (IFs).


SEQUENCE LISTING (14 sequences)

SEQ ID NO: 1 - DNA, 660 bp, Salmonella typhimurium
atcaacatcaggccaaatgccgtcagtatcagcgaacgttgctccagtccaatctgtcctttgggatccagcagcgcagaattacagccactgagtaatgcagtgcctgcaattaatgacaaccatcccaaacttttattgtatttcctgagtctcatttaacgacctcaattccacgggacctggtggcgtttaaagtgtaggggcattttacggtaaggttacattactgtaaacatgattggttctgtgttaccaagttctgccgcgcatgtcacatatgttgccaagtatgtcgaaataactttaagacactgttgaaactttgcgttataacggatgaggtctgtaagtgggatgttgagggataattggtataaccaatgtaaaaataaaacaattacttaacaatttattatcaataaaaggacaatcaacggcagaaatttaaatttaatcggctgaatcgtgtaaagaaaataggtagcgtagaaattaccaataatttccaaaattctgttgcgcacaaaaagacaaatatattttttatcaattgatggttatttagccgttataattccgggtcgttattacaacggcaaataatgttttacagcaggcgtgtttttcgtagcgccaggtagtccagcaacccgccaa

SEQ ID NO: 2 - DNA, 509 bp, Salmonella typhimurium
tccaatctgtcctttgggatccagcagcgcagaattacagccactgagtaatgcagtgcctgcaattaatgacaaccatcccaaacttttattgtatttcctgagtctcatttaacgacctcaattccacgggacctggtggcgtttaaagtgtaggggcattttacggtaaggttacattactgtaaacatgattggttctgtgttaccaagttctgccgcgcatgtcacatatgttgccaagtatgtcgaaataactttaagacactgttgaaactttgcgttataacggatgaggtctgtaagtgggatgttgagggataattggtataaccaatgtaaaaataaaacaattacttaacaatttattatcaataaaaggacaatcaacggcagaaatttaaatttaatcggctgaatcgtgtaaagaaaataggtagcgtagaaattaccaataatttccaaaattctgttgcgcacaaaaagacaaatatattttttatcaattg

SEQ ID NO: 3 - DNA, 259 bp, Salmonella typhimurium
gtgttaccaagttctgccgcgcatgtcacatatgttgccaagtatgtcgaaataactttaagacactgttgaaactttgcgttataacggatgaggtctgtaagtgggatgttgagggataattggtataaccaatgtaaaaataaaacaattacttaacaatttattatcaataaaaggacaatcaacggcagaaatttaaatttaatcggctgaatcgtgtaaagaaaataggtagcgtagaaattaccaataattt

SEQ ID NO: 4 - DNA, 20 bp, Artificial Sequence
  uscA1 primer
atcaacatcaggccaaatgc

SEQ ID NO: 5 - DNA, 20 bp, Artificial Sequence
  uscA2 primer
taataacgacccggaattat

SEQ ID NO: 6 - DNA, 24 bp, Artificial Sequence
  uscA3 primer
tagcccgggctcgagtccaatctg

SEQ ID NO: 7 - DNA, 24 bp, Artificial Sequence
  uscA4 primer
acggctaaataagcttcaattgat

SEQ ID NO: 8 - DNA, 21 bp, Artificial Sequence
  uscA5 primer
aatctgtcctttggaattcag

SEQ ID NO: 9 - DNA, 21 bp, Artificial Sequence
  uscA6 primer
gcaacagaaggatccaaatta

SEQ ID NO: 10 - DNA, 24 bp, Artificial Sequence
  uscA7 primer
taacgacctgaattccacgggacc

SEQ ID NO: 11 - DNA, 21 bp, Artificial Sequence
  uscA8 primer
ataccaaggatccctcaacat

SEQ ID NO: 12 - DNA, 24 bp, Artificial Sequence
  uscA9 primer
tggtaacacggatccaatcatgtt

SEQ ID NO: 13 - DNA, 24 bp, Artificial Sequence
  uscA10 primer
aacatgattgaattcgtgttacca

SEQ ID NO: 14 - DNA, 30 bp, Artificial Sequence
  pRS415 vector primer
attaactgcgcgtcgccgctttcatcggtt